SEQUENCE LISTING (37 sequences)

SEQ ID NO: 1 - DNA, 19 bp, Artificial sequence
  Single strand DNA oligonucleotide
gacagtcagccgcatcttc

SEQ ID NO: 2 - DNA, 19 bp, Artificial sequence
  Single strand DNA oligonucleotide
cgttgactccgaccttcac

SEQ ID NO: 3 - DNA, 24 bp, Artificial sequence
  Single strand DNA oligonucleotide
actggagagtgctgaagattgatg

SEQ ID NO: 4 - DNA, 31 bp, Artificial sequence
  Single strand DNA oligonucleotide
ctacttcctctgtgatgttgtattttttaag

SEQ ID NO: 5 - DNA, 20 bp, Artificial sequence
  Single strand DNA oligonucleotide
tgacacctgggtggaaagag

SEQ ID NO: 6 - DNA, 20 bp, Artificial sequence
  Single strand DNA oligonucleotide
ccactgtggtcccatctaag

SEQ ID NO: 7 - DNA, 20 bp, Artificial sequence
  Single strand DNA oligonucleotide
ccatactgagggaccaactg

SEQ ID NO: 8 - DNA, 20 bp, Artificial sequence
  Single strand DNA oligonucleotide
ggctgctcttgtaggaagtg

SEQ ID NO: 9 - DNA, 20 bp, Artificial sequence
  Single strand DNA oligonucleotide
tgtctttcctggcactaacg

SEQ ID NO: 10 - DNA, 20 bp, Artificial sequence
  Single strand DNA oligonucleotide
aaacagtccaggccagtatg

SEQ ID NO: 11 - DNA, 20 bp, Artificial sequence
  Single strand DNA oligonucleotide
gtctgctagccaggatccac

SEQ ID NO: 12 - DNA, 20 bp, Artificial sequence
  Single strand DNA oligonucleotide
gcttccacatgtcctcacaa

SEQ ID NO: 13 - DNA, 20 bp, Artificial sequence
  Single strand DNA oligonucleotide
tctgaggggagaaatcctga

SEQ ID NO: 14 - DNA, 20 bp, Artificial sequence
  Single strand DNA oligonucleotide
ggaagagatggccaaaatga

SEQ ID NO: 15 - DNA, 20 bp, Artificial sequence
  Single strand DNA oligonucleotide
atggaccttcaccagaccta

SEQ ID NO: 16 - DNA, 20 bp, Artificial sequence
  Single strand DNA oligonucleotide
tgggttgaggagagaaacac

SEQ ID NO: 17 - DNA, 18 bp, Artificial sequence
  Single strand DNA oligonucleotide
agccgggatctaccatac

SEQ ID NO: 18 - DNA, 18 bp, Artificial sequence
  Single strand DNA oligonucleotide
tcatggctaccacttgac

SEQ ID NO: 19 - DNA, 20 bp, Artificial sequence
  Single strand DNA oligonucleotide
gctgctctgggattctcttc

SEQ ID NO: 20 - DNA, 19 bp, Artificial sequence
  Single strand DNA oligonucleotide
tggcgagctcaggtacttc

SEQ ID NO: 21 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 22 - DNA, 2575 bp, Homo sapiens
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 23 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETRLADWIHSSGGWAEFTALYGDGALEEARRLREGNWASVRTVLTGAVALGALVTVGAFFASK

SEQ ID NO: 24 - DNA, 3621 bp, Homo sapiens
atcacccggcgccgggccctccctctgcccccccctttctcctccctccttccttccctcccttcctccctctctccctccctcccagctcctgcaccaggaaacggcccggatcccggcagcggcctgacccggctccacgctggccaggaggatgaaaggccccagctgggggctccttgccaccagtgctgtgtcttaagagctgccatcccggctggccgcccggatggcgaccccagcctcggccccagacacacgggctctggtggcagactttgtaggttataagctgaggcagaagggttatgtctgtggagctggccccggggagggcccagcagctgacccgctgcaccaagccatgcgggcagctggagatgagttcgagacccgcttccggcgcaccttctctgatctggcggctcagctgcatgtgaccccaggctcagcccaacaacgcttcacccaggtctccgatgaactttttcaagggggccccaactggggccgccttgtagccttctttgtctttggggctgcactgtgtgctgagagtgtcaacaaggagatggaaccactggtgggacaagtgcaggagtggatggtggcctacctggagacgcggctggctgactggatccacagcagtgggggctgggcggagttcacagctctatacggggacggggccctggaggaggcgcggcgtctgcgggaggggaactgggcatcagtgaggacagtgctgacgggggccgtggcactgggggccctggtaactgtaggggccttttttgctagcaagtgaaagtccagggccaggtggggctaggtgtggctgggggccaggagagcaggaacagaacagagaaatgcccttggaagaagtggagttggtggatgggtgggcatggaacaggatgggcagagaaagggtagtgtgtgagggagctgagtaggccaggtaggcgattggaagagtgagcaggacacagaggggaggggaatgttttggcaagtttaggggcacaggagatgtagtcgttccagggctgggggaggtgggagggatcacgcctataggtgtgggcacatgaaacgacctggaacttgcttcacagccctgaggaaggtggacttacataagcagctgtattccattagatgagtgggatttagggaacgcagaaggcacatccctttggaatggaagcttaggggttctcaggtgatagggagaggtggctgttaacagtgggctgcttggacacgcgtgtgcatgtgcacgcatgctggtgtgcatgctgggctgcctggcaaatctggtggtgatgggattcctcaaggagaaaacattccctcttgcaatggcaagaactaggggcagttctctgtccctcctcccaacccctcctttcccctgcccttgtcctgatgcctcaaggcttagagagaaacattgtatccagaccgagggctctgctgcttctttccagaaagtgattggcaaggctttggagagaagagcagttctgcagctggccttgttccttcatcatcccccttccttgtgcattatgcacttgctgctgcctcctgggctctgatagaagggcagggctgttgagcctggatgggtggaggcttaggtagccggacctgcctgccaccctcctctcccactcaggcacaatggtgcctaaagtgtttccaatctctgggacctctgtacccaaactgaaactctaaattggggccctaactaattttccttttgaggttgtgggcataagtgctgatctagaatacagtctgggtcccacactgtgtctcagtgagactgttgatgccttgagatgaccatttcagatctgaatcccatgggtgtgagggtgatgggtactccaggactggcctatgctgtgttgtgggctttggttcggctttatcaggggccaggcatatgggttctagagtacctaccatgacctagaagcatttatgatttatttgaagccacactgtttgcatgggtgttacttgtctgtacctcagagtctgaggatgttaactttggaactcgcagtcctctagaacagcttcagattatggctttttcttttgaggaagaaattattcactccagatgcatgccctgagccagacctcactgctgcactttccaaggtgctaagattgctgctctccaatgctaactttctgacacagtgctctagaaccctgcctgtggtcctgagcactgatcaccttagctagaccatggttgactcttcttggagattttcacttggtcctagaatgtggcaacgtagttgtgctcgccagaacgtgggaccaaattggcctcaggtgttgagtccagacttctgcttttgagagagggctgcactttttcatggtatttctaggggaggtggtaggctgcatgtgccacttggtcttgttgtgagtatgctgacaccagaaactcagagccagcttgtggcaagcagttggggtggggggtctctgacttgctcaggacaaactaggccagtggttttcaaactgcttggcagagccctgaagtttcctaggggttgcctcaggagtccttggggagatgaagggggtggggagctgagcaggctgggcaatttgccctcaaacagaacagctccccttgtagctgtcttacatattggggttcagggtaagattttatttgcattaaggggtttgctgctgaaaaaaagttggaaaaccactgactagaccatcggctccaaattggagtctgtgcttccttccccaggtatggagcacactcttcaccctaccctctaccacaggacacatatccctgttagcattccccgggacctttagccaagaggagctgcagggaccatggccaggttaccaaaatgccctgctctgaagccttgacacctgggtggaaagagaggctgttttctgaaagggtaaagggcttggtctggattcccagaagcatagcttagatgggaccacagtgggcaattttgacctgtcctgcccttcttagcttgaagggaaaccccagagactcttctgtcagggaaaactagggactctcttctagagccatatagttccttgggattagctcttggccaagaaggctgagtatggttcccaatttttaaatccatttcattttttaaaaaataagggaaataaatgtaattgccatttttcaaagattaagtaggaggagaggggtttcttgctctccagagcccaaagggacaaatagggactttgtttaggccaaggaaggagcggaagtagggcaactcggtcctgcgattattaatcccactccccacttattctagggcacacaaacactattttacttttttaaaatcataaaacggcagaacagatttggttagtttagaagaaaagaaagctctataaatataaatctatattcctgtatttttatttaataatttataaataccaagttcatttgacttttatttttgtgtaatatgtaatgatcgtattaaaaacaataaataaagcccagaagtttaatgagaaggactgaaaaaaaaaaaaaaaaaa

SEQ ID NO: 25 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 26 - DNA, 2175 bp, Homo sapiens
gttgtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaaa

SEQ ID NO: 27 - RNA, 9 nt, Artificial sequence
  Example of a sequences that can be used to form the shRNA loop
uucaagaga

SEQ ID NO: 28 - RNA, 9 nt, Artificial sequence
  Example of a sequences that can be used to form the shRNA loop
uuuguguag

SEQ ID NO: 29 - DNA, 19 bp, Artificial sequence
  Bcl-xl siRNA target sequence
cagggacagcatatcagag

SEQ ID NO: 30 - DNA, 19 bp, Artificial sequence
  Bcl-xl siRNA target sequence
ggagactagattgcctttg

SEQ ID NO: 31 - DNA, 21 bp, Artificial sequence
  Bcl-xl siRNA target sequence
ggcaggcgacgagtttgaact

SEQ ID NO: 32 - DNA, 21 bp, Artificial sequence
  Bcl-w siRNA target sequence
ctcggtcctgcgattattaat

SEQ ID NO: 33 - DNA, 20 bp, Artificial sequence
  Bcl-w siRNA target sequence
aagtgcaggagtggatggtg

SEQ ID NO: 34 - DNA, 21 bp, Artificial sequence
  Bcl-w siRNA target sequence
aagggttatgtctgtggagct

SEQ ID NO: 35 - DNA, 20 bp, Artificial sequence
  CDKN1A - cyclin-dependent kinase inhibitor 1A (p21, Cip1) siRNA target sequence
cttcgactttgtcaccgaga

SEQ ID NO: 36 - DNA, 21 bp, Artificial sequence
  CDKN1A - cyclin-dependent kinase inhibitor 1A (p21, Cip1) siRNA target sequence
cagtttgtgtgtcttaattat

SEQ ID NO: 37 - DNA, 21 bp, Artificial sequence
  CDKN1A - cyclin-dependent kinase inhibitor 1A (p21, Cip1) siRNA target sequence
ctggcattagaattatttaaa

(19) United States
(12) Patent Application Publication
(10) Pub. No.: US 2018/0345047 A1
(43) Pub. Date: Dec. 6, 2018

(54) METHOD OF PROMOTING HAIR GROWTH

(71) Applicants: Yeda Research and Development Co. Ltd., Rehovot (IL); Yissum Research Development Company of the Hebrew University of Jerusalem Ltd., Jerusalem (IL)

(72) Inventors: Valery KRIZHANOVSKY, Rehovot (IL); Reut YOSEF, Rehovot (IL); Noam PILPEL, Rehovot (IL); Ittai BEN-PORATH, Jerusalem (IL); Liat DASSA, MaAle Adumim (IL); Ronit AMIEL-TOKARSKY, Modiln (IL)

(21) Appl. No.: 15/552,506

(22) PCT Filed: Feb. 25, 2016
(86) PCT No.: PCT/IL2016/050220

§ 371 Date: Aug. 22, 2017

Related U.S. Application Data
(60) Provisional application No. 62/120,959, filed on Feb. 26, 2015.

(51) Int. Cl. A61P 17/14; A61Q 7/00; A61K 9/00

(52) U.S. Cl. CPC A61P 17/14; A61K 31/11; A61K 9/0014; A61Q 7/00

(57) ABSTRACT

A method of promoting hair growth or reducing hair loss in a subject is disclosed. The method comprises contacting the scalp and skin region in which there is desire for hair growth of the subject with an effective amount of at least one agent which down-regulates an activity and/or an amount of a protein selected from the group consisting of p21, Bcl-xL and Bcl-w. Compositions capable of same are also disclosed.

### FIELD AND BACKGROUND OF THE INVENTION

The present invention, in some embodiments thereof, relates to a method of promoting hair growth by the down-regulation of genes encoding Bcl-2-family proteins and/or p21.

Cellular senescence, a stable form of cell cycle arrest, is a mechanism limiting the proliferative potential of cells. Senescence can be triggered in many cell types in response to diverse forms of cellular stress. It is a potent barrier to tumorigenesis and contributes to the cytotoxicity of certain anti-cancer agents. While senescence limits tumorigenesis and tissue damage in a cell autonomous manner, senescent cells induce inflammation, tissue aging, tissue destruction and promote tumorigenesis and metastasis in a cell non-autonomous manner. Therefore, their elimination might lead to tumor prevention and inhibition of tissue aging. Indeed, elimination of senescent cells was shown to slow down tissue aging in an animal model (Baker et al., 2011).

Organisms might have developed elaborate mechanisms to eliminate senescent cells in order to avoid their deleterious effects on the microenvironment. However, their fate in tissue is not well characterized. On the one hand, benign melanocytic nevi (moles) are highly enriched for senescent cells, yet they can exist in skin for a lifetime, implying that senescent cells can be stably incorporated into tissues. On the other hand, it has been previously shown that components of the innate immune system specifically recognize and eliminate senescent cells in vitro and target senescent cells in vivo leading to tumor regression and reversion of liver fibrosis (Krizhanovsky et al., 2008b; Sagiv et al., 2012; Xue et al., 2007). Therefore, senescent cells can turn over in vivo and the immune system contributes to this turnover. The effort that the immune system invests in recognition and elimination of senescent cells suggests, although not directly, that senescent cells are deleterious for the organism and their elimination is beneficial.

In the last decade multiple studies identified the genes and the pathways required for senescence induction or bypass of the senescence phenotype. Two tumor suppressor pathways, controlled by the p53 (TP53) and p16INK4a (CDKN2A), regulate senescence response. p53 promotes senescence by transactivating genes that inhibit proliferation, while p16INK4a, accompanied by the p53 target p21 (CDKN1A), inhibit cyclin-dependent kinases (CDKs) 2 and 4, thereby preventing pRB phosphorylation and promoting repressive heterochromatin formation to silence proliferation-associated genes.

Bcl-2-family proteins play a central role in cell death regulation and are capable of regulating diverse cell death mechanisms that encompass apoptosis, necrosis and autophagy (Cory et al., 2003; Reed, 2008). The function of the founding member of the family, Bcl-2, in senescence remains controversial. It was proposed to be either upregulated or downregulated in senescent cells and was associated with either negative or positive regulation of apoptosis of these cells (Uraoka et al., 2011; Wang, 1995). In addition to Bcl-2, the family includes the anti-apoptotic proteins Bcl-xL, Bcl-w, Mcl-l and A1, and is intensively studied as a target for pharmacological intervention in cancer (Azmi et al., 2011; Zeitlin et al., 2008).

U.S. Patent Application No. 20120189539 teaches a chemical which down-regulates Bcl-xL for the treatment of cancer.

U.S. Patent Application No. 20040001811 teaches pharmaceutical compositions comprising dsRNA targeted against Bcl-2 family members for the treatment of cancer.

U.S. Patent Application No. 20070258952 teaches administration of siRNA targeted against numerous genes including Bcl-xL and p-21.

U.S. Patent Application No. 20110301192 teaches administration of chemical agents that down-regulate p-21 for the treatment of cancer.

### SUMMARY OF THE INVENTION

According to an aspect of some embodiments of the present invention there is provided a method of promoting hair growth or reducing hair loss in a subject comprising contacting the scalp and skin region in which there is desire for hair growth of the subject with an effective amount of at least one agent which down-regulates an activity and/or an amount of a protein selected from the group consisting of p21, Bcl-xL and Bcl-w, thereby promoting hair growth or reducing hair loss in the subject.

According to an aspect of some embodiments of the present invention there is provided a method of promoting hair loss or removal in a subject comprising administering to the subject an effective amount of at least one agent which up-regulates an activity and/or an amount of a protein selected from the group consisting of p21, Bcl-xL and Bcl-w, thereby promoting hair loss or removal in the subject.

According to an aspect of some embodiments of the present invention there is provided a method of treating alopecia in a subject in need thereof, the method comprising administering to the subject a therapeutically effective amount of at least one agent which down-regulates an activity and/or an amount of a protein selected from the group consisting of p21, Bcl-xL and Bcl-w, thereby treating the alopecia.

According to an aspect of some embodiments of the present invention there is provided a use of at least one agent which down-regulates an activity and/or an amount of a protein selected from the group consisting of p21, Bcl-xL and Bcl-w for the treatment of alopecia.

According to an aspect of some embodiments of the present invention there is provided a method of reducing hair loss in a subject who is required to undergo a treatment or procedure that results in damage to the hair follicle, comprising administering to the subject an effective amount of at least one agent which down-regulates an activity and/or an amount of a protein selected from the group consisting of p21, Bcl-xL and Bcl-w prior to or concomitant with the treatment or procedure, thereby reducing hair loss in the subject.

According to an aspect of some embodiments of the present invention there is provided a hair product comprising at least one agent which down-regulates an activity and/or an amount of a protein selected from the group consisting of p21, Bcl-xL and Bcl-w.

According to some embodiments of the invention, the skin region is the eyebrow region.

According to some embodiments of the invention, the at least one agent down-regulates an activity and/or an amount of both Bcl-xL and Bcl-w.

According to some embodiments of the invention, the at least one agent is a chemical agent.

According to some embodiments of the invention, the at least one agent is a polynucleotide agent directed against a polynucleotide encoding the protein.

According to some embodiments of the invention, the chemical agent is selected from the group consisting of ABT-737, ABT-263, Gossypol, AT-101, TW-37 and Obatoclax.

According to some embodiments of the invention, the polynucleotide agent is an siRNA.

According to some embodiments of the invention, the subject has alopecia.

According to some embodiments of the invention, the subject has a reduced amount of hair due to chemotherapy.

According to some embodiments of the invention, the subject has a reduced amount of hair due to an environmental factor.

According to some embodiments of the invention, the at least one agent down-regulates an activity and/or an amount of both Bcl-xL and Bcl-w.

According to some embodiments of the invention, the at least one agent is a chemical agent.

According to some embodiments of the invention, the at least one agent is a polynucleotide agent directed against a polynucleotide encoding the protein.

According to some embodiments of the invention, the chemical agent is selected from the group consisting of ABT-737, ABT-263, Gossypol, AT-101, TW-37 and Obatoclax.

According to some embodiments of the invention, the polynucleotide agent is an siRNA.

According to some embodiments of the invention, the at least one agent is comprised in a composition formulated for topical administration.

According to some embodiments of the invention, the topical composition is selected from the group consisting of a shampoo, a foam, a lotion, a serum, a gel, a film-forming drug, a hair conditioner, a paste, a mousse, a cream, a spray and a powder.

According to some embodiments of the invention, the treatment is chemotherapy.

According to some embodiments of the invention, the hair product is selected from the group consisting of a shampoo, a foam, a lotion, a serum, a gel, a film-forming drug, a hair conditioner, a paste, a mousse, a cream, a spray and a powder.

According to some embodiments of the invention, the administering comprises topically administering.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-J illustrate that inhibition of Bcl-2 family members by ABT-737 eliminates senescent cells in the skin and induces stem cell proliferation.

(A) SA-β-Gal stain (blue) of skin sections from K5-rtTA/tet-p14 mice treated for 4 weeks with doxycycline to activate p14^{ARF} in the epidermis, and subsequently treated with ABT-737 (p14+ABT) or vehicle (p14+V) for 4 consecutive days. Sibling mice carrying only the tet-p14 transgene (Ctrl) were used as negative controls. (B) Mean number of SA-β-Gal-positive cells per microscopic field in control, vehicle treated and ABT-737 treated mice. Values indicate mean±S.E.M. across individual mice (dots). (C) Representative FACS analyses of SA-β-Gal activity in epidermal cells isolated from indicated mice, using the fluorescent substrate C₁₂FDG, which provides increased sensitivity relative to section staining. Gate indicates SA-β-Gal⁺ cell percentage. FSC—forward scatter. (D) Skin sections of p14^{ARF}-expressing and control mice after 2 day treatment with ABT-737 or vehicle, stained for the human transgenic p14^{ARF} protein (white, arrows) and for keratin 14 (K14, green) marking the basal epidermal layer. (E) p14-positive cell numbers per field in the indicated mice. Values indicate mean±S.E.M. across individual mice (dots). (F) Sections of same mice stained for the apoptosis marker cleaved caspase-3 (CC3). Arrows indicate apoptotic cells in a section from an ABT-737-treated mouse. (G) CC3-positive cell number per field in the indicated mice. Values indicate mean±S.E.M. across individual mice (dots). (H) Representative hair-follicle bulge sections of p14-expressing mice after 4 days of ABT-737 or vehicle treatment, stained for the proliferation marker Ki67 (green) and the bulge marker K15 (red). Arrows indicate Ki67⁺ bulge stem cells in ABT-737 treated mice. (I) Mean numbers of Ki67+K15+ cells per follicle in individual p14-expressing mice after 4 days of ABT-737 or vehicle treatment, scored by image analysis. Values indicate mean±S.E.M. across individual mice (dots); >15 fields were scored in each mouse. (J) FACS analysis of epidermal cells obtained from indicated mice after 2 days of ABT-737 or vehicle treatment, stained for CD34, CD49f and Sca1. Plots show only Sca1-negative cell fraction, enriched for follicle-epidermal cells; purple gate indicates percentage of CD34⁺/CD49f^{high} hair-follicle stem cells. In all panels *−P <0.05; **−P <0.005; ***−P <0.0005.

FIGS. 2A-B illustrate that inhibition of Bcl-2 family members by ABT-737 on the expression of K15 and Ki67.

(A) Sections of hair follicle bulges stained for the bulge marker K15 (red) and the proliferation marker Ki67 (green) from p14^{ARF}-expressing mice treated with ABT-737 for 2 days, and sacrificed 3 days subsequent to the last treatment. (B) Number of Ki67-positive cells in individual bulges from ABT-737 treated (n=3) and vehicle treated (n=2) mice. Dots indicate individual follicles from scored mice, bars indicate mean±s.e.m. *P <0.05 by Student's t-test.

### DESCRIPTION OF SPECIFIC EMBODIMENTS OF THE INVENTION

The present invention, in some embodiments thereof, relates to a method of killing senescent cells by the down-regulation of genes encoding Bcl-2-family proteins and/or p21 for the promotion of hair growth.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The methods of the present invention relate to a method of regulating hair growth on the body. The hair may be body hair, facial hair (e.g. eyebrow, eyelashes, etc) or head hair.

In one aspect, the present invention relates to a method of promoting hair growth by down-regulating an activity and/or an amount of a protein selected from the group consisting of p21, Bcl-xL and Bcl-w.

In another aspect, the present invention relates to reducing (e.g. preventing) hair loss. This aspect may be particular useful in subjects who are about to undergo treatments (such as chemotherapy) which are known to affect the hair follicle.

The methods described herein are suitable to counteract the effects of normal aging, exposure to damaging agents such as chemo/radiation and others, and pathological conditions in which hair is lost.

The term “Bcl-xL” refers to the human protein also known as B-cell lymphoma-extra large, having a sequence as set forth in SEQ ID NO: 21 and homologs and orthologs thereof. The cDNA sequence of human Bcl-xL is set forth in SEQ ID NO: 22.

The term “Bcl-w” refers to the human protein also known as Bcl-2-like protein 2, having a sequence as set forth in SEQ ID NO: 23 and homologs and orthologs thereof. The cDNA sequence of human Bcl-w is set forth in SEQ ID NO: 24.

The term “p21” also known as “cyclin-dependent kinase inhibitor 1” refers to the human protein having a sequence as set forth in SEQ ID NO: 25 and homologs and orthologs thereof. The cDNA sequence of human p21 is set forth in SEQ ID NO: 26.

According to a particular embodiment, the method comprises down-regulation of Bcl-xL and Bcl-w.

According to another embodiment, the method comprises down-regulation of each of Bcl-xL, Bcl-w and p21.

According to still another embodiment, the method comprises down-regulation of p-21 and down-regulation of Bcl-xL.

According to still another embodiment, the method comprises down-regulation of p-21 and down-regulation of Bcl-w.

As used herein, the phrase “downregulating an activity and/or amount” of a target protein refers to a downregulation of at least 10%, at least 20%, at least 30%, at least 40%, at least 50% at least 60%, at least 70%, at least 80% or even at least 90% of the activity and/or amount of the target protein. In addition, the term “downregulating” may also refer to full inhibition.

Downregulation of Bcl-xL and/or Bcl-w and/or p21 can be effected using chemical agents. Chemical agents known to decrease the activity of Bcl-xL and/or Bcl-w include ABT-737, ABT-263, Gossypol, AT-101, TW-37 and Obatoclax.

According to a particular embodiment, the agent is ABT-737 or ABT-263.

ABT-737 and ABT-263 (ABT-263 being a bioavailable form called “Novatoclax”, Abbot) are currently in Phase II for multiple myeloma, lymphoma, acute leukemia, CLL, small cell lung cancer.

Gossypol (natural) Phase II/III for head and neck tumors, pancreatic cancer.

AT-101 (Gossypol derivative; Ascenta Therapeutics) Phase II/III for pancreatic cancer, head and neck cancer, glioma.

TW-37 (Uni Michigan) Phase II for pancreatic cancer, lymphoma.

Obatoclax (GX15-070MS; Gemin X, later Cephalon, now Teva) Phase II for myeloma, myelofibrosis and mantle cell lymphoma.

An example of a chemical agent which down-regulates activity of p21 is disclosed in U.S. Patent Application No. 20110301192, incorporated herein by reference.

Other compositions for down-regulating senescent cells which may be administered together with the compositions described herein are described in WO2014089124, WO2013152038 and WO 2013152041, the contents of which are incorporated herein by reference.

Downregulation of Bcl-xL and/or Bcl-w and/or p21 can also be effected on the genomic and/or the transcript level using a variety of molecules which interfere with transcription and/or translation (e.g., RNA silencing agents, Ribozyme, DNAzyme and antisense), or on the protein level using e.g., antagonists, enzymes that cleave the polypeptide and the like.

Following is a list of agents capable of downregulating expression level and/or activity of Bcl-xL and/or Bcl-w and/or p21.

One example, of an agent capable of downregulating Bcl-xL and/or Bcl-w and/or p21 is an antibody or antibody fragment capable of specifically binding thereto. Preferably, the antibody is capable of being internalized by the cell.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Downregulation of Bcl-xL and/or Bcl-w and/or p21 can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In particular, the present invention also contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433 and Diallo et al, Oligonucleotides, Oct. 1, 2003, 13(5): 381-392. doi:10.1089/154545703322617069.

The present invention also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

It will be appreciated that more than one siRNA agent may be used to target Bcl-xL or Bcl-w and/or p21.

Thus, the present invention contemplates use of at least two siRNAs that target Bcl-xL, at least three siRNAs that target Bcl-xL, or even at least four siRNAs that target Bcl-xL, each targeting a different sequence in the Bcl-xL gene.

Further, the present invention contemplates use of at least two siRNAs that target Bcl-w, at least three siRNAs that target Bcl-w, or even at least four siRNAs that target Bcl-w, each targeting a different sequence in the Bcl-w gene.

Further, the present invention contemplates use of at least two siRNAs that target p21, at least three siRNAs that target p21, or even at least four siRNAs that target p21, each targeting a different sequence in the p21 gene.

The strands of a double-stranded interfering RNA (e.g., a siRNA) may be connected to form a hairpin or stem-loop structure (e.g., a shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (SEQ ID NO: 27; Brummelkamp, T. R. et al. (2002) Science 296: 550) and 5′-UUUGUGUAG-3′ (SEQ ID NO: 28; Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to a miRNA, rather than triggering RNA degradation.

Synthesis of RNA silencing agents suitable for use with the present invention can be effected as follows. First, the Bcl-xL and/or Bcl-w mRNA and/or p21 sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level.

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, a suitable siRNA capable of downregulating Bcl-xL can be the siRNA of SEQ ID NO: 29, 30 or 31. A suitable siRNA capable of downregulating Bcl-w can be the siRNA of SEQ ID NO: 32, 33 or 34. A suitable siRNA capable of downregulating p21 can be the siRNA of SEQ ID NO: 35, 36 or 37.

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

Another agent capable of downregulating Bcl-xL or Bcl-w or p21 is a DNAzyme molecule capable of specifically cleaving an mRNA transcript or DNA sequence thereof. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis (Itoh et al, 20002, Abstract 409, Ann Meeting Am Soc Gen Ther www(dot)asgt(dot)org). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of Bcl-xL or Bcl-w or p21 can also be effected by using an antisense polynucleotide capable of specifically hybridizing with an mRNA transcript encoding Bcl-xL, Bcl-w or p21.

Design of antisense molecules which can be used to efficiently downregulate Bcl-xL or Bcl-w or p21 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published [Matveeva et al., Nature Biotechnology 16: 1374 - 1375 (1998)].

Another agent capable of downregulating Bcl-xL or Bcl-w or p21 is a ribozyme molecule capable of specifically cleaving an mRNA transcript encoding Bcl-xL or Bcl-w or p21. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms has demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

An additional method of regulating the expression of Bcl-xL or Bcl-w or p21 genes in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence of Bcl-xL or Bcl-w or p21 regulatory region, a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

The present invention further contemplates up-regulating an amount and/or activity of Bcl-xL and/or Bcl-w and/or p21 in promote hair loss.

Thus, according to yet another aspect of the present invention there is provided a method of promoting hair loss or removal in a subject comprising administering to the subject an effective amount of at least one agent which up-regulates an activity and/or an amount of a protein selected from the group consisting of p21, Bcl-xL and Bcl-w, thereby promoting hair loss or removal in the subject.

In one embodiment the agent which up-regulates the activity and/or amount of p21, Bcl-xL or Bcl-w is a polynucleotide agent that encodes the protein.

Polynucleotide agents for down-regulating or up-regulating an amount or activity of Bcl-xL and/or Bcl-w and/or p21 may be administered as part of an expression construct. In this case, the polynucleotide agent is ligated in a nucleic acid construct under the control of a cis-acting regulatory element (e.g. promoter) capable of directing an expression of the agent capable of down-regulating or up-regulating Bcl-xL and/or Bcl-w and/or p21 in a constitutive or inducible manner.

The nucleic acid agent may be delivered using an appropriate gene delivery vehicle/method (transfection, transduction, etc.). Optionally an appropriate expression system is used. Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www(dot)Invitrogen(dot)com).

The expression construct may also be a virus. Examples of viral constructs include but are not limited to adenoviral vectors, retroviral vectors, vaccinia viral vectors, adeno-associated viral vectors, polyoma viral vectors, alphaviral vectors, rhabdoviral vectors, lenti viral vectors and herpesviral vectors.

A viral construct such as a retroviral construct includes at least one transcriptional promoter/enhancer or locus-defining element(s), or other elements that control gene expression by other means such as alternate splicing, nuclear RNA export, or post-transcriptional modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably, the signal sequence for this purpose is a mammalian signal sequence or the signal sequence of the peptide variants of the present invention. Optionally, the construct may also include a signal that directs polyadenylation, as well as one or more restriction site and a translation termination sequence. By way of example, such constructs will typically include a 5′ LTR, a tRNA binding site, a packaging signal, an origin of second-strand DNA synthesis, and a 3′ LTR or a portion thereof.

Preferably the viral dose for infection is at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles.

Double stranded RNA may be synthesized by adding two opposing promoters to the ends of the gene segments, wherein one promoter is placed immediately 5′ to the gene and the opposing promoter is placed immediately 3′ to the gene segment. The dsRNA may then be transcribed with the appropriate polymerase.

The application of small polynucleotide agents (e.g. siRNAs) as potential therapeutic agents requires delivery approaches that will enhance their pharmacological properties. These delivery approaches aim to: (1) increase the retention time of the small polynucleotide agents in the circulatory system by reducing the rate of renal clearance; (2) protect the small polynucleotide agents from serum nucleases; (3) ensure effective biodistribution; (4) facilitate targeting to and uptake of the small polynucleotide agents into the target cells; and (5) promote trafficking to the cytoplasm and uptake into RISC. A variety of approaches have been developed that promote small polynucleotide agent delivery in vivo, including cationic nanoparticles, lipids and liposomes, antibody (Ab)-fusion molecules [Ab-protamine and Ab-poly-arginine, as well as cholesterol and aptamer-conjugated agents. On their own, small polynucleotide agents such as siRNAs fall below the size threshold for renal filtration and are rapidly cleared from the circulatory system. Complexes of small polynucleotide agents and the various delivery reagents remain in the circulation for longer, either because they exceed the size cut-off for renal clearance or because the delivery agents promote association with serum proteins (e.g. serum albumin). In addition, the encapsidation of the small polynucleotide agents into nanoparticles (using either lipid- or cationic-polymer-based systems) helps to shield them from serum nucleases. Ab-fusion molecules have been used to effectively deliver naked, unmodified small polynucleotide agents to specific cell types following intravenous injection. Although the siRNAs are thought to be exposed on the surface of these recombinant Ab-fusion molecules, they were effectively delivered to the target cells, suggesting that complexation with these molecules provides some protection from nucleolytic degradation. The incorporation of chemical modifications to the phosphate backbone, the sugar moiety and the nucleoside bases of the small polynucleotide agents increases its resistance to degradation by serum nucleases. As some of these modifications are detrimental to the silencing efficacy, however, a balance must be maintained between the incorporation of chemical modifications and the inhibitory activity of the small polynucleotide agents. An attractive strategy for decreasing the dosage of the small polynucleotide agents needed to achieve effective silencing and minimizing off-target silencing in bystander cells is the use of delivery agents that target the small polynucleotide agents to specific cell types and tissues. This has been achieved using Abs or ligands that are fused to highly positively charged peptides or proteins, with which the small polynucleotide agents can associate by electrostatic interactions, or by directly conjugating aptamers or ligands to the small polynucleotide agents. These reagents (Abs, ligands and aptamers) can bind with high affinity to cell-surface molecules and deliver the small polynucleotide agents specifically to cells expressing these markers. By combining these targeting reagents with nanoparticles (e.g. immunoliposomes containing lipid nanoparticles coated with specific Abs), the quantity of small polynucleotide agents delivered and, as a consequence, the efficacy of silencing can be increased.

Accordingly, the present invention contemplates use of lipid-based systems for the delivery of these agents. Useful lipids for lipid-mediated transfer of the gene are, for example, DOTMA, DOPE, and DC-Chol [Tonkinson et al., Cancer Investigation, 14(1): 54-65 (1996)]. Recently, it has been shown that Chitosan can be used to deliver nucleic acids to the intestine cells (Chen J. (2004) World J Gastroenterol 10(1):112-116). Other non-lipid based vectors that can be used according to this aspect of the present invention include but are not limited to polylysine and dendrimers, carbon nanotubes, nanogels, polymer based particles.

As used herein, the term “subject” refers to a mammalian subject, preferably a human.

The agents of the present invention (and combinations thereof) may be provided per se or may be formulated in compositions intended for a particular use. It will be appreciated that combinations of the agents described herein may be provided in a single formulation or may be provided in individual compositions.

Contemplated compositions include those that comprise an agent which downregulates of Bcl-xL and an agent which downregulates Bcl-w (e.g. siRNA agents).

Another contemplated composition is one which includes an agent which downregulates of Bcl-xL and an agent which downregulates Bcl-w (e.g. siRNA agents) and an agent which downregulates p21 (e.g. siRNA agent).

Another contemplated composition is one which includes an agent which downregulates of Bcl-xL and Bcl-w (e.g. chemical agent) and an agent which downregulates p21 (e.g. siRNA agent).

Another contemplated composition is one which includes an agent which downregulates of Bcl-xL and Bcl-w (e.g. chemical agent) and an agent which downregulates p21 (e.g. chemical agent).

Further, the present inventors contemplate providing combinations of the agents individually packed in a single article of manufacture.

Thus, one contemplated article of manufacture includes an agent which downregulates of Bcl-xL and an agent which downregulates Bcl-w (e.g. siRNA agents).

Another contemplated article of manufacture is one which includes an agent which downregulates of Bcl-xL and an agent which downregulates Bcl-w (e.g. siRNA agents) and an agent which downregulates p21 (e.g. siRNA agent).

Another contemplated article of manufacture is one which includes an agent which downregulates of Bcl-xL and Bcl-w (e.g. chemical agent) and an agent which downregulates p21 (e.g. siRNA agent).

Another contemplated article of manufacture is one which includes an agent which downregulates of Bcl-xL and Bcl-w (e.g. chemical agent) and an agent which downregulates p21 (e.g. chemical agent).

The agents of the present invention may be formulated for cosmetics.

Such compositions typically comprise pharmaceutically acceptable excipient, notably dermatologically acceptable suitable for external topical application.

The cosmetic composition according to the present invention may further comprise at least one pharmaceutical adjuvant known to the person skilled in the art, selected from thickeners, preservatives, fragrances, colorants, chemical or mineral filters, moisturizing agents, thermal spring water, etc.

Any number of complete hair care products may be included as a hair care composition of the present exemplary hair care composition including, but in no way limited to, shampoo, conditioner, anti curl lotion, anti humectant pomade, color enhancing conditioner, color glaze, color mousse, color treated hair conditioner, colored hair shampoo, corrective styling mousse, cover gray, curl defining gel, curl defining shampoo, dandruff shampoo, defining cream, detanglers, ethnic conditioner, ethnic relaxer, ethnic shampoo, foam mask, foaming pomade, foaming styler, gel, hair gloss, hair loss shampoo, hydrating masque, straighteners, keratin treatment, tonic, molding cream, non-permanent hair color, pre-shampoo treatment, protecting spray, regrowth treatment, relaxing serum, restructuring serum, root lift, root pump, sculpting gel, shine pomade, silk therapy, smoothing cream, smoothing mask, smoothing serum, straightening balm, strengtheners, thickening lotion, vitamins, volume booster, volumizing conditioner, and/or volumizing gel.

In the case of color ingredients, a solvent, such as peroxide, may be incorporated with the use of the hair care composition. For example, peroxide may be added during rehydration of the hair care composition, or the peroxide or other solvent may be incorporated with the color so as to activate when exposed to water or some other solvent.

The present exemplary hair care composition can include any number of natural or organic ingredients including, but in no way limited to, aloe derivatives, aloe barbadensis gel, alpha lipoic acid, aleurites muluccana seed oil, ascorbyl palmitate, apricot kernel oil, aqua, basil, behentrimonium methosulfate, calendula extract, chamomile extract, castor oil, carnauba, cetyl alcohol, citronellol, coumarin, citric acid, sodium sweetalmondamphoacetate, geranium oil, hydrolyzed wheat protein, jojoba oil, kelp, kelp extract, lanolin, macadamia oil, palmitic acid, panthenol (pro Vitamin B5), rosemary extract, safflower oil, shea butter, sodium ascorbyl phosphate, sorbitol, stearic acid, sucrose stearate, teatree extract, and/or tocopheryl acetate.

Additionally, the present exemplary hair care composition can include Acetamide MEA, Alcohol, Algae Extract, Algal Polysaccharides, Allantoin, AMP, Ammonium Lauryl Sulfate, Amphoteric Surfactants, Annatto Extract-annionic Surfactants, Beet Extract-Benzophenone, Beta Carotene, Biotin, Boric Acid, Butylene Glycol, Caramel, Carbomer 940, Carrageenan, Cationic Surfactants, Ceteareth-5, Cetearyl Alcohol, Ceteth-2, Ceteth-20, Cetrimonium Bromide, Cetrimonium Chloride, Cetyl Alcohol, Cetyldimonium Chloride, Chloroxylenol, Cocamide DEA, Cocamide MEA, Cocamidopropyl Betaine, Coco Betaine, Cyclomethicone, DEA Oleth-3 Phosphate, DEA Oleth-10 Phosphate Diazolidinyl, Dicetyldimonium Chloride, Dimethicone, Dimethicone Copolyol, Dimethyl Lauramine Isostearate, Dimethyl Stearamine, EDTA Ethyl Ester PVM/MA Copolymer, Essential Oils, Glyceryl Monstearate, Glyceryl Stearate, Glycolic Acid, Glycol Stearate, Grapeskin Extract, Green Tea Extract, Guar Hydroxypropyltrimonium Chloride, Hyaluronic Acid, Hydrolyzed Human Hair Keratin Protein, Hydroxyethel Cellulose, Hydroxypropyl Methylcellulose, Isobutane, Isopropanol, Isopropyl Alcohol (Isopropyl Palmitate, Lactamide MEA Lactic Acid, Laureth-3, Lecithin, Lineolamido Propyl Ethydimonium Ethosulfate, Magnesium Citrate, Methacryloyl Ethyl Betaine Methylchloroisthiazolinone, Methylisothiazolinone, Methyl Paraben, Myristalkonium Chloride, Niacinamide, Nonionic Surfactants, Nonoxynol 12-O, Cresol, Octylacrylamide Acrylate Butylaminoethyl Methacrylate Copolymer, Octylacrylamide Butylaminoethyl Methacrylate Copolymer, Octyl Methoxycinnamate, Oleth 20, Orange Peel Extract, Palm Kernelamide DEA and MEA Panthenol, PEG, Pentacrythritol Tetra Caprate/Caprylate, Phenyl Trimethicone, Polyquaternium 11, Polysorbate 20, Polysorbate 80, Potassium Sorbate, PPG 2 Isodeceth 12, Pristane, Propane, Propyl Paraben, Propylene Glycol Dicocoate, Pyroxidine HCL, Quaternium 15, Salicylic Acid, SD 40 Alcohol, SD Alcohol 40B, Shea Butter, Sodium Cetyl Sulfate, Sodium Hydroxymethylglycinate, Sodium Laureth Sulfate, Sodium Myristoyl Sarcosinate, Sodium PCA, Sodium Thiosulfate, Sorbitol, Stearalkonium Chloride, Stearamidopropyl Dimethyamine, Steareth 21, Stearic Acid, Stearyl Alcohol, Surfactant, TEA Laureth Sulfate, TEA Lauryl Sulfate, Tetrasodium EDTA, Triethanolamine (TEA) and Xanthan Gum. Further, the present exemplary hair care composition can include a scented polymer to provide a desired scent to the hair care composition.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Mouse experiments: K5-rtTA/tet-p14, described in (Toakrsly-Amiel et al.) were held on a mixed C57B1/129sv background. Mice received doxycycline (2 mg/ml) in the drinking water at 3 weeks of age for activation of the p14ARF transgene. Sibling tet-p14 single transgene control mice received doxycycline for the same period. After four weeks of transgene activation, ABT-737 (75 mg/kg in 30% propylene glycol, 5% Tween 80, 3.3% dextrose in water pH 4-5) or vehicle was injected into p14ARF-expressing mice intraperitoneally for 2 or 4 consecutive days. Mice were then shaved, sacrificed and back skins were paraffin embedded for immunohistology or frozen in OCT solution for cryosectioning and SA-β-gal stains. Equal numbers of males and females were used in the vehicle and ABT-737 treated groups and showed similar responses.

Immunohistology: Immunohistology was performed according to standard procedures on 5 μm paraffin sections, using Peroxidase Substrate kits (Vector) or fluorescently labeled secondary antibodies (Jackson). Antibodies used: p14ARF (Abcam), CC3 (Cell Signaling), Ki67 (Labvision) and K15 (Santa Cruz). For Senescence-associated β-Galactosidase (SA-β-gal) stains, 10-12 μm cryosections of OCT-embedded mouse skins were fixed in 0.5% glutaraldehyde for 15 minutes, stained over night at 37° C. with 40 mM phosphate buffer pH=6 with 5 mM K4Fe(CN)6, 5 mM K3Fe(CN)6, 150 mM NaCl, 2 mM MgCl2 and 1 mg/ml X-gal, washed in PBS, fixed in 95% ethanol for 15 minutes, counterstained with nuclear fast red, dehydrated and mounted. Bright field images were collected using an Olympus CX41 and DS-Fi1 camera and processed using NIS Elements software (Nikon). Fluorescent images were taken using an Olympus FV1000 confocal microscope. Positive cells were scored from >10 microscopic fields in each sample either by direct microscopic observation (SA-β-gal, p14) or on collected images (all other stains).

### Example 1

### Inhibition of Bcl-2 Family Members Eliminates Senescent Cells in the Skin and Leads to Re-Activation of Hair Follicle Stem Cells

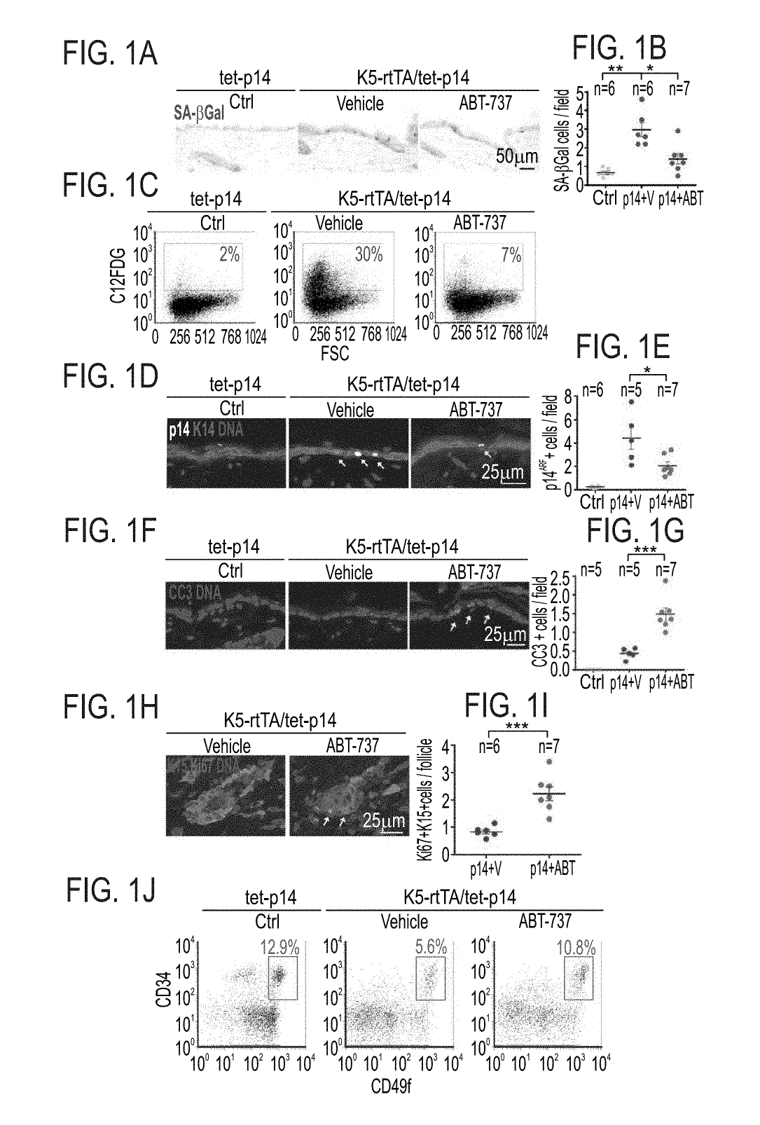

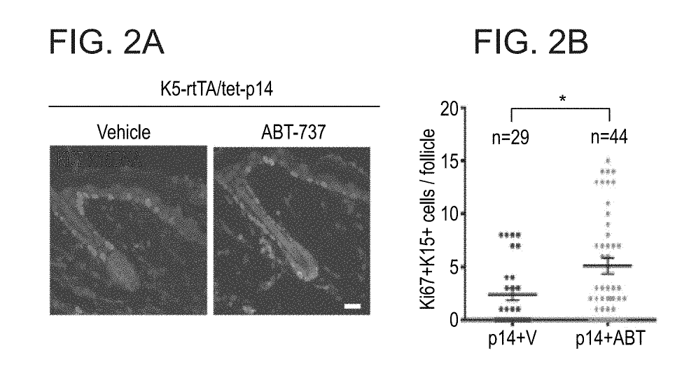

The present inventors set out to test whether the survival signals provided by Bcl-2 family members are necessary for the survival and retention of senescent cells in tissues. To do this, they used double transgenic K5-rtTA/tet-p14 mice, in which the human p14^{ARF} gene is inducibly expressed in the basal layer of the skin epidermis. Induction of p14^{ARF} in these mice activates p53 and generates senescent epidermal cells that are retained in the tissue for weeks. To generate senescent cells, expression of p14^{ARF} was activated in 3-week-old mice for a period of 4 weeks, and then the mice were treated with ABT-737 for 4 consecutive days. The number of senescent cells in the epidermis, determined by SA-β-Gal staining, was dramatically reduced in the ABT-737-treated mice relative to vehicle-treated mice (FIGS. 1A-C). A similar degree of elimination was observed after ABT-737 treatment of these mice for 2. Concomitantly, the percentage of epidermal cells in which the transgenic p14^{ARF} protein could be detected was reduced (FIGS. 1D-E), indicating preferred elimination of transgene-expressing cells. Increased levels of apoptosis were detected in the epidermis after 2 days of ABT-737 treatment (FIGS. 1F-G), consistent with increased apoptosis as the mechanism of senescent cell elimination. These findings indicate that the survival signal provided by BCL family proteins is an essential component for the ability of senescent cells to be retained in the tissue, and in its absence they rapidly die.

Upon p14^{ARF} activation in mice at 3 weeks of age nearly all hair follicles are arrested in the resting (telogen) state, and only rare stem cells in the bulge express the proliferation marker Ki67³⁶ (FIGS. 5H-I). Interestingly, we found that treatment with ABT-737 after 2-4 weeks of p14^{ARF} induction led to an increase in the numbers of proliferating hair-follicle stem cells in the bulge (FIGS. 5H-I). This effect was also evident three days after treatment was stopped (FIGS. 2A-B). Furthermore, the number of CD34⁺/CD49f^{high} bulge stem cells was increased following treatment (FIG. 5J). These findings suggest that the elimination of senescent hair-follicle stem cells allows other (non-senescent) bulge cells to initiate proliferation and repopulate the stem cell compartment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of promoting hair growth or reducing hair loss in a subject having age-related alopecia or chemically induced alopecia comprising contacting the scalp and/or skin region in which there is desire for hair growth of the subject with an effective amount of at least one agent which down-regulates an activity and/or an amount of a protein selected from the group consisting of p21, Bcl-xL and Bcl-w, thereby promoting hair growth or reducing hair loss in the subject.

2-3. (canceled)

4. The method of claim 1, wherein said agent down-regulates an activity and/or an amount of both Bcl-xL and Bcl-w.

5. The method of claim 1, wherein said agent is a chemical agent, with the proviso that the chemical agent is not Gossypol.

6. The method of claim 1, wherein said agent is a polynucleotide agent directed against a polynucleotide encoding said protein.

7. The method of claim 5, wherein said chemical agent is ABT-737 or ABT-263.

8. The method of claim 6, wherein said polynucleotide agent is an siRNA.

9. (canceled)

10. The method of claim 1, wherein the subject has a reduced amount of hair due to chemotherapy.

11. The method of claim 1, wherein the subject has a reduced amount of hair due to an environmental factor.

12-18. (canceled)

19. The method of claim 1, wherein said at least one agent is comprised in a composition formulated for topical administration.

20. The method of claim 19, wherein the topical composition is selected from the group consisting of a shampoo, a foam, a lotion, a serum, a gel, a film-forming drug, a hair conditioner, a paste, a mousse, a cream, a spray and a powder.

21-32. (canceled)
